(19) United States
(12) Patent Application Publication
(10) Pub. No.: US 2005/0106256 A1
(43) Pub. Date: May 19, 2005

(54) Composition of composite hydrogel microparticles containing polysaccharide or derivatives thereof and method for preparing the same

(75) Inventors: Yu-Chuen Hung, Taoyuan Hsien (TW); Chi-Shu Wei, Pan Chiao City (TW); Lien-Hua Chiu, Taipei Hsien (TW); Wen-Yen Chiu, Taipei City (TW); Chia-Jen Wen, Taipei City (TW)

Correspondence Address: DANIEL B. SCHEIN, P. O. BOX 28403, SAN JOSE, CA, 95159, US

(73) Assignee: Taiwan Textile Research Institute

(21) Appl. No.: 10/968,283

(22) Filed: Oct. 18, 2004

(30) Foreign Application Priority Data
Nov. 17, 2003 (TW) 92132185

(51) Int. Cl. A61K 9/14

(52) U.S. Cl. 424/488

(57) ABSTRACT

A composition of composite hydrogel microparticles containing polysaccharide or derivatives thereof and a method for preparing the same are disclosed. The method utilizes an ionic initiator to initiate a dispersion polymerization of a hydrogel monomer, and then performs a composite reaction with polysaccharide or derivatives thereof carrying opposite charges, so as to form composite hydrogel microparticles containing polysaccharide or derivatives thereof.

### RELATED APPLICATIONS

The present application is based on, and claims priority from, Taiwan Application Serial Number 92132185, filed Nov. 17, 2003, the disclosure of which is hereby incorporated by reference herein in its entirety.

### FIELD OF THE INVENTION

The present invention relates to a method for preparing composite hydrogel microparticles, and more particularly, to a composition of composite hydrogel microparticles containing polysaccharide or derivatives thereof and a method for preparing the same.

### BACKGROUND OF THE INVENTION

Biomedical materials are in urgent need of rapid development as the related industry faces internal transformation. Hydrogel, with its diverse varieties as developed in Europe, the United States and Japan, is likely to be an area with promising growth.

Hydrogel is applied extensively in biomedical fields, such as wound dressings, hemostatic materials, drug controlled release system, and even implants used in plastic surgery and artificial intraocular lens for implanting into eyeballs. Because the key development of the biomedical materials towards functional medical products with high additional value, hydrogel will be an important material, especially for artificial skins, wound dressings, cell and tissue cultures, face masks, and the like.

In U.S. Pat. No. 6,486,213, Chen et al. disclose hydrogels for drug delivery, which contain a pH-sensitive polymer component grafted with a temperature-sensitive block polymer component, and then mixed with one or more drugs for drug delivery.

In U.S. Pat. No. 5,834,007, Kubota discloses a temperature-sensitive hydrogel polymer, which exists in a gel-state at a temperature higher than the lower critical solution temperature (LCST), and a sol-state at a temperature lower than the LCST. An antibacterial agent and a wound healing-accelerator are dissolved or dispersed in the hydrogel polymer of the sol-state to form a wound-covering material.

In U.S. Pat. No. 4,956,350, Mosbey discloses a wound-filling composition comprising a polysaccharide chitosan effective to promote healing, provide anti-microbial activity, or both, at least one compatible hydrocolloid material and water. Preferably, compositions applied to a dermal ulcer exhibit wound healing capability without syneresis, and are able to absorb wound exudate.

In addition, in U.S. Pat. No. 5,420,197, Lorenz et al. disclose a dermatologically-compatible composition comprising a hydrophilic gel which comprises a blend of a neutralized chitosan and a hydrophilic poly (N-vinyl lactam). The product is used in, for example, wound dressings, burn dressings, drug delivery dressings, cosmetic mask dressings, and the like.

In addition to wound dressings, the hydrogel can be useful in hemostatic material. For example, in T.W. Pat. No. 394,689, Bozigian et al. disclose an absorbent dressing for absorbing exudate from a wound. The absorbent dressing contains a plurality of dried absorbent hydrogel particles sealed within a porous container, and the hydrogel particles are used for absorbing the exudate and a wound-healing agent.

The manufacture of the hydrogel disclosed in the prior arts requires a two-stage process, in which the hydrogel polymer is formed first, and then the hydrogel is mixed with or absorbs active components in medical treatment, such as chitosan or drugs. Moreover, the method for producing the hydrogel polymer in the prior arts is to form a block hydrogel polymer, but not hydrogel particles with uniform particle size.

Further, the hydrogel applied in the dressings are mainly selected from a temperature-sensitive or pH-sensitive hydrogel polymer. The temperature-sensitive hydrogel polymer is used because it exists in a gel-state at a temperature higher than the LCST and a sol-state at a temperature lower than LCST. At a temperature lower than the LCST, an antibacterial agent and a wound healing-accelerator are dissolved or dispersed in the hydrogel polymer of the sol-state, and then the gel-state hydrogel polymer is formed into a wound-covering material with absorbed active components having a medicating effect when the temperature is raised above the LCST. Additionally, the pH-sensitive hydrogel polymer is used because it has different properties in different pHs to achieve the goal of absorbing the active components.

### SUMMARY OF THE INVENTION

In consideration of disadvantages of the hydrogel polymer in the prior preparations, it is an object of the present invention to provide a composition of hydrogel microparticles and a method for preparing the same, for preparing composite hydrogel microparticles containing polysaccharide or derivatives thereof.

It is another object of the present invention to provide a method for preparing hydrogel microparticles, which method utilizes an ionic initiator to perform a dispersion polymerization of a hydrogel monomer or its prepolymer in the presence of polysaccharide or derivatives thereof, for synthesizing composite hydrogel microparticles having environmental sensitivity and biodegradability.

It is another object of the present invention to provide a method for preparing hydrogel microparticles, which method performs a dispersion polymerization by using an ionic initiator to start the hydrogel monomer or prepolymer, and then adds polysaccharide or derivatives thereof with charges opposite that of the initiator, so as to perform the polymerization of hydrogel without using an emulsifier or a detergent.

It is still another object of the present invention to provide a method for preparing hydrogel microparticles, which method utilizes a single process to prepare a hydrogel polymer having polysaccharide, so as to save time in processing.

It is yet another object of the present invention to provide a method for preparing hydrogel microparticles, which method utilizes a dispersion polymerization to provide a resultant hydrogel polymerization product having hydrogel particles with uniform particle size. In application, the resultant product may be in various forms of a liquid, lotion, gel, and, after drying, a powder or film.

According to the aforementioned objects of the present invention, the present invention provides a method for preparing hydrogel microparticles, which performs a dispersion polymerization by using an ionic initiator, such as ammonium persulfate, potassium persulfate, or 2,2′-azobis(2-methylprionamidine)dihydrochloride, and optionally adding a suitable amount of a crosslinking agent, to initiate the hydrogel monomer or its prepolymer. Simultaneously, to the reaction solution is added the polysaccharide or derivatives thereof with charges opposite that of the initiator. Polysaccharide or derivatives thereof with positive (or negative) charges existing in the reaction solution can attract the hydrogel polymer with negative (or positive) charges to form composite hydrogel microparticles during the process of the dispersion polymerization. The polysaccharide or derivatives thereof may be alginic acid, chitin, chitosan, collagen, hyaluronic acid or derivatives thereof.

The polysaccharide or derivatives thereof applied in the present invention, due to carrying charges, can also work as an emulsifier or a detergent. Thus, the polysaccharide or derivatives thereof not only gives the hydrogel extra additional abilities, such as, anti-microbial, deodorization, or hemostasis, but also adjusts the surface potential of hydrogel microparticles by using the concentration of the composite polysaccharide or derivatives thereof, so as to increase stability of the hydrogel to prevent coagulation, and further to control the particle size of the hydrogel microparticles.

In comparison with related techniques in the past, the process of composite hydrogel of the invention is a single process, not a two-stage process that prepares an absorbing material of hydrogel first and covers the desired polysaccharide with the resultant hydrogel. Additionally, the prior techniques mostly provide block hydrogel absorbent material. The process of the invention is a dispersion polymerization, and thus the polymerized product is hydrogel particles with uniform particle size. In application, the resultant product may be in various forms of a liquid, lotion, gel, and, after drying, a powder or film. Moreover, the process of the invention, which prepares the composite hydrogel containing the polysaccharide or derivatives thereof, not only gives the hydrogel added functions, but also preserves the original functions of the hydrogel.

The object of the invention is to combine the hydrogel with the polysaccharide or derivatives thereof in the reacting stage to form composite hydrogel microparticles containing the polysaccharide or derivatives thereof, and to make hydrogel that has a particular function and structure. Also, hydrogel can exist in various forms, including a liquid, lotion, and gel, as a result of controlling the amount of water in reaction, and, after drying, exists as a powder or film. Therefore, composite hydrogel microparticles containing polysaccharide or derivatives thereof prepared by the method of the invention is less restricted in application as, for example, wound dressings, hemostatic materials, or drug controlled release systems.

The method for preparing composite hydrogel microparticles containing polysaccharide or derivatives thereof of the invention performs a dispersion polymerization by mixing an ionic initiator, a hydrogel monomer or its prepolymer, a polysaccharide or derivatives thereof with water, at a temperature in the range of about 10° C. to 100° C. for about 1 to 5 hours, to form composite hydrogel microparticles containing the polysaccharide or derivatives thereof. The polysaccharide or derivatives thereof may be alginic acid, chitin, chitosan, collagen, hyaluronic acid or derivatives thereof. The polysaccharide or derivatives thereof may carry charges opposite that of initiator, and the polysaccharide or derivatives thereof without charges, which can be treated by an acidic or alkaline substance, may carry positive or negative charges. The acidic substance may be an inorganic or organic acid, and the alkaline substance may be an inorganic or organic alkali.

To the solution of the hydrogel monomer or its prepolymer may be further added a suitable amount of a crosslinking agent, a divinyl reactive monomer or its prepolymer or bisacrylamide, to control the particle size. The content of the crosslinking agent is preferably in the range of about 0 to 30 weight percent (wt %) of the hydrogel monomer or derivatives thereof. The content of polysaccharide or derivatives thereof is preferably in the range of about 5 to 60 wt % of the hydrogel monomer or derivatives thereof. The polysaccharide or derivatives thereof can work as an emulsifier or a detergent, and thus can control the particle sizes of hydrogel microparticles containing polysaccharide or derivatives thereof prepared by the present method. The hydrogel monomer or the prepolymer thereof are, for example, polymethacrylic acid and ester derivatives thereof containing a hydroxyl group, polyacrylamide, polymethacrylamide and a derivative thereof, poly-N-vinyl-2-pyrrolidone and polyvinyl alcohol.

The composite hydrogel microparticles containing polysaccharide or derivatives thereof, prepared by the method disclosed in the present invention, have been covered with or have absorbed polysaccharide or derivatives thereof during dispersion polymerization of the monomer. The composite hydrogel microparticles of the present invention are also environmentally sensitive to allow them to combine more components with a medicating or curing effect.

Hence, for clarifying the method for preparing composite hydrogel microparticles containing polysaccharide or derivatives thereof of the invention, the ratio and reaction condition of each reactant used in the reaction, and various forms and particle sizes of composite hydrogel microparticles containing polysaccharide or derivatives thereof according to various reaction conditions, can be clearly understood in accordance with preferred embodiments of the invention.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 depicts a diagram of particle size distribution showing composite hydrogel microparticles containing polysaccharide or derivatives thereof prepared by using various ratios of N-isopropyl acrylamide monomer and chitosan according to the present invention;

FIG. 2 depicts a diagram of particle size distribution showing composite hydrogel microparticles containing polysaccharide or derivatives thereof prepared by using the same ratio of N-isopropyl acrylamide monomer and chitosan but various concentrations of the crosslinking agent according to the present invention; and

FIG. 3 depicts a diagram of particle size distribution showing composite hydrogel microparticles containing polysaccharide or derivatives thereof prepared by using the same ratio of N-isopropyl acrylamide monomer and chitosan but various concentrations of the crosslinking agent according to the present invention.

### DETAILED DESCRIPTION OF THE PREFERRED EMBODIMENT

Examples 1 to 3 illustrate production of composite hydrogel microparticles containing polysaccharide or derivatives thereof by using the ratio of each reactant or different reaction conditions, according to the method for preparing composite hydrogel microparticles containing polysaccharide or derivatives thereof disclosed by the present invention. Examples 4 to 6 disclose particle size distributions of composite hydrogel microparticles containing polysaccharide or derivatives thereof, which are prepared by using various ratios of various reactants, as detected by light scattering. It is noted that using various ratios of reactants can effectively control the desired composite hydrogel microparticles as shown in the results of the particle size distribution.

### EXAMPLE 1

A solution of 7 g N-isopropyl acrylamide (NIPAAM) monomer, 0.7 g N,N′-methylene bisacrylamide (MBA), 1.4 g chitosan, 1000 g deionized water, and 3 g glacial acetic acid, is stirred at room temperature in a 2 L-reaction flask. MBA works as a crosslinking agent, and glacial acetic acid can make chitosan carry positive charges.

After stirring well, the solution is heated to 50° C. while stirring at 300 rpm. To this stirred solution is added an anionic initiator. The reaction is continued for 3 hours. The resultant product is liquid composite hydrogel microparticles.

### EXAMPLE 2

A solution of 7 g NIPAAM monomer, 0.21 g MBA, 1.4 g chitosan, 350 g deionized water, and 3 g glacial acetic acid is stirred at room temperature in a 1 L-reaction flask. MBA works as the crosslinking agent, and glacial acetic acid can make chitosan carry positive charges.

After stirring well, the solution is heated to 50° C. while stirring at 300 rpm. To this stirred solution is added the anionic initiator. The reaction is continued for 3 hours to form liquid composite hydrogel microparticles. After drying, the resultant product further becomes a powder or film.

### EXAMPLE 3

A solution of 7 g NIPAAM monomer, 0.35 g MBA, 2.1 g chitosan, 350 g deionized water, and 3 g glacial acetic acid is stirred at room temperature in a 1 L-reaction flask. MBA works as the crosslinking agent, and glacial acetic acid is used for making chitosan to carry positive charges.

After stirring well, the solution is heated to 50° C. while stirring at 300 rpm. To this stirred solution is added the anionic initiator. The reaction is continued for 3 hours to form gelled composite hydrogel microparticles.

### EXAMPLE 4

Example 4 discloses the effect on changing the ratios of reactants, chitosan and NIPAAM monomer.

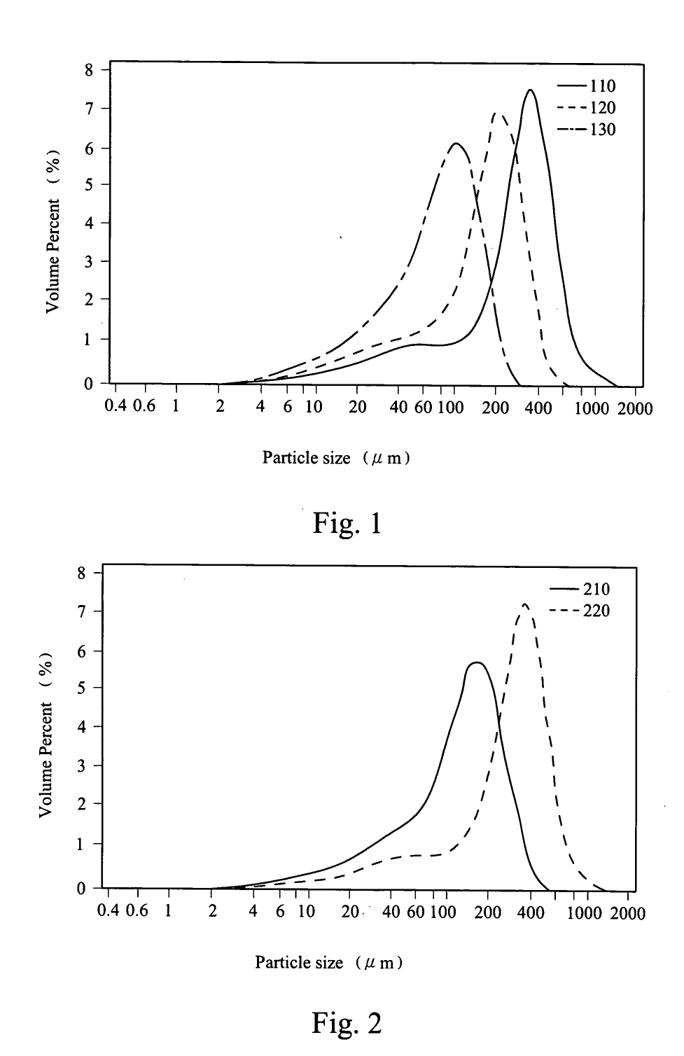

Reference is made to FIG. 1, which depicts a diagram of particle size distribution showing composite hydrogel microparticles containing polysaccharide or derivatives thereof prepared by using various ratios of NIPAAM monomer and chitosan. A horizontal axis represents the particle size (μm) and a vertical axis represents the volume percent of hydrogel microparticles in water.

In FIG. 1, about 9 wt % of composite hydrogel microparticles containing chitosan prepared by using the ratios of reactants chitosan to NIPAAM monomer, which are 1:10, 2:10, and 3:10, respectively, are analyzed by light scattering in water. The curve 110 represents the light scattering result of composite hydrogel microparticles containing chitosan prepared with a ratio of 1:10 of chitosan to NIPAAM monomer, the curve 120 represents the light scattering result of composite hydrogel microparticles containing chitosan prepared with a ratio of 2:10 of chitosan to NIPAAM monomer, and the curve 130 represents the light scattering result of composite hydrogel microparticles containing chitosan prepared with a ratio of 3:10 of chitosan to NIPAAM monomer.

As shown in FIG. 1, the particle sizes of composite hydrogel microparticles, which are prepared by using the method disclosed by the invention, are smaller on swelling when the ratio of chitosan to NIPAAM monomer is more. The reason is that chitosan carrying charges not only is used for a reactant, but also plays a role in a detergent in the polymerization disclosed by the invention. The effect of the detergent with the more amount of chitosan is better, and the resultant composite hydrogel microparticles have smaller and more uniform particle size.

Accordingly, the particle size of the product, composite hydrogel microparticles containing polysaccharide or derivatives thereof, can be effectively controlled by changing the ratio of polysaccharide used in the polymerization, so as to satisfy requirements of subsequent applications.

### EXAMPLE 5

Example 5 discloses the effect of changing the concentration of the crosslinking agent in the reactants.

Reference is made to FIG. 2, which depicts a diagram of particle size distribution showing composite hydrogel microparticles containing polysaccharide or derivatives thereof prepared by using the same ratio of NIPAAM monomer and chitosan but various concentrations of the crosslinking agent. A horizontal axis represents the particle size (μm) and a vertical axis represents the volume percent of hydrogel microparticles in water.

In FIG. 2, about 9 wt % of composite hydrogel microparticles containing chitosan, which are prepared with a ratio of 1:10 of reactants chitosan to NIPAAM monomer but various concentrations of the crosslinking agent, are analyzed by light scattering in water. The curve 210 represents the light scattering result of composite hydrogel microparticles containing chitosan prepared by utilizing the crosslinking agent in usage of 3% of NIPAAM monomer, and the curve 220 represents the light scattering result of composite hydrogel microparticles containing chitosan prepared by utilizing the crosslinking agent in usage of 5% of NIPAAM monomer.

As shown in FIG. 2, the particle size of composite hydrogel microparticles is smaller after swelling when the concentration of the crosslinking agent is greater. One reason is that when the number of nuclei with a higher concentration of crosslinking agent is greater, the polymerized particles are smaller in the whole polymerization system. The other reason is that the crosslinking degree and the restriction of the polymerized hydrogel particles with the higher concentration of the crosslinking agent are stronger, and the swelling degree in water is lower. Therefore, the detected particle size is smaller.

### EXAMPLE 6

Example 6 is based on the result of Example 5, and observes that the effect on changing the concentration of the crosslinking agent in the reactants is caused by changing the ratios of reactants, chitosan and NIPAAM monomer.

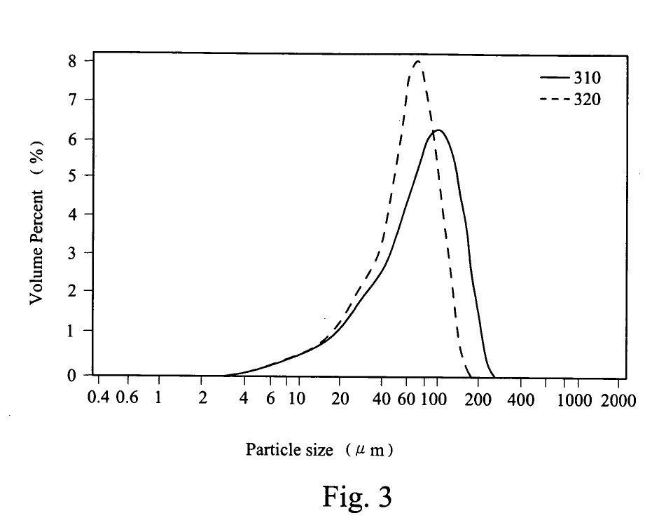

Reference is made to FIG. 3, which depicts a diagram of particle size distribution showing composite hydrogel microparticles containing polysaccharide or derivatives thereof prepared by using the same ratio of NIPAAM monomer and chitosan but various concentrations of the crosslinking agent. A horizontal axis represents the particle size (μm) and a vertical axis represents the volume percent of hydrogel microparticles in water.

In FIG. 3, about 9 wt % of composite hydrogel microparticles containing chitosan, which are prepared by using a ratio of 3:10 of reactants chitosan to NIPAAM monomer and a concentration of the crosslinking agent the same as that used in Example 5, are analyzed by light scattering in water. The curve 310 represents the light scattering result of composite hydrogel microparticles containing chitosan prepared by utilizing the crosslinking agent in usage of 3% of NIPAAM monomer, and the curve 320 represents the light scattering result of composite hydrogel microparticles containing chitosan prepared by utilizing the crosslinking agent in usage of 5% of NIPAAM monomer.

As shown in FIG. 3, there is a coincidence effect on the concentration of the crosslinking agent; in other words, the particle size of composite hydrogel microparticles is smaller after swelling when the concentration of the crosslinking agent is higher. But in comparison with FIG. 2, the effect on changing the concentration of the crosslinking agent is significantly reduced. It is hypothesized that the particle sizes of composite hydrogel microparticles prepared by using the higher ratios of reactants chitosan to NIPAAM monomer are originally smaller, as shown in the result of Example 5, and thus the effect on particles with the smaller particle sizes is reduced depending on changing the concentration of the crosslinking agent.

Hence, changing the ratio of polysaccharide and the concentration of the crosslinking agent used in the polymerization can effectively control the particle sizes of composite hydrogel microparticles containing polysaccharide or derivatives thereof, so as to satisfy the need in subsequent applications.

As illustrated by the results of the above examples, the method for preparing composite hydrogel microparticles containing polysaccharide or derivatives thereof provided by the invention certainly can prepare composite hydrogel microparticles containing polysaccharide or derivatives thereof. Advantages of applying the method provided by the invention are as follows:

1. When hydrogel is performed with a dispersion polymerization, polysaccharide or derivatives thereof with charges as an emulsifier or a detergent, and an initiator with charges opposite to that of polysaccharide or derivatives thereof, can perform the dispersion polymerization of hydrogel. The hydrogel and the polysaccharide or derivatives thereof form composite hydrogel microparticles containing polysaccharide or derivatives thereof depending on the principle that hydrogel has charges opposite to those of polysaccharide or derivatives thereof. The invention, which prepares hydrogel microparticles through the dispersion polymerization, not only eliminates the extra cost of the detergent and the emulsifier, but also avoids the difficulty of removing the emulsifier from the end product. Furthermore, the effect of composite hydrogel microparticles containing polysaccharide or derivatives thereof, which are prepared by the method provided by the invention, not only gives the hydrogel extra additional abilities, such as anti-microbial, deodorization, hemostasis, and so on, but also has the functions of the emulsifier and the detergent. Additionally, the surface potential of hydrogel microparticles can be adjusted by using the content of the composite polysaccharide or derivatives thereof, so as to increase stability of hydrogel and prevent coagulation, and further to control the particle size of hydrogel microparticles.

2. The process of composite hydrogel provided by the invention is a single process, not a two-staged process that prepares an absorbing material of hydrogel first and covers or absorbs the desired polysaccharide with the resultant hydrogel. Thus, the composite hydrogel has composite functions of hydrogel and polysaccharide, and the processing time can be conserved.

3. In comparison with the prior processes of hydrogel polymerization, which are mostly producing techniques of forming block hydrogel absorbing materials, the preparation method provided by the invention is a dispersion polymerization, and thus the polymerized product is hydrogel particles with uniform particle size for use in various forms of liquid, lotion, gel, and after drying, a powder or film.

4. The composite hydrogel microparticles prepared according to the technique contents of the invention, not only keep the advantage of hydrogel, but also have the properties of polysaccharide or derivatives thereof. For example, composite microparticles formed with hydrogel and chitosan, not only have the properties of hydrogel, such as self-adhesion, moist wound healing, and semi-permeable membrane, but also have the properties of chitosan. Therefore, when the hydrogel touches the wound directly, it brings many advantages, for example, the wound may be healed quickly, neither hurt nor be caused secondary injury when changing dressings, microbes cannot penetrate through the hydrogel, and the frequency of dressing changes may be reduced. Also, the hydrogel can activate lymphocytes and macrophages to kill microbes, prevent the wound surface from suppurating, and facilitate angiogenesis and granulation.

5. Composite hydrogel microparticles prepared by the method of the invention have fewer restrictions in applications, such as wound dressings, hemostatic materials, drug controlled release system, and the like.

Composite hydrogel microparticles containing polysaccharide or derivatives thereof prepared by the method of the invention can be applied in biomedical industries, medical device industries and the like. Because development for biomedical materials in the future is towards functional medical products with high additional value, how to effectively and quickly obtain composite hydrogel microparticles containing polysaccharide or derivatives thereof with uniform particle size which are controllable for supplying usage of artificial skins, wound dressings, cell and tissue cultures, face masks, and the like will be very important. The method provided by the invention can satisfy the above needs for effectiveness, quickness, uniformity of particle size, and particle size. Further, in the future, hydrogel combined with Chinese herbal medicine for application in cosmetics and dressings is an important application field for hydrogel polymer. It can be understood that the method for preparing composite hydrogel microparticles containing polysaccharide or derivatives thereof disclosed by the invention indeed has infinite business potential.

It can be known from the aforementioned preferred embodiment of the present invention, as is understood by a person skilled in the art, the foregoing preferred embodiments of the present invention are illustrated of the present invention rather than limiting of the present invention. It is intended to cover various modifications and similar arrangements included within the spirit and scope of the appended claims, the scope of which should be accorded the broadest interpretation so as to encompass all such modifications and similar structure.

1. A composition of composite hydrogel microparticles containing polysaccharide or derivatives thereof, comprising:
an ionic initiator;
a hydrogel monomer or a prepolymer thereof;
a polysaccharide or derivatives thereof; and
water;
wherein the ionic initiator applied to a polymerization of the hydrogel monomer or the prepolymer thereof carries a first charge, the polysaccharide or the derivatives thereof carries a second charge, the first charge is opposite to the second charge, and a ratio of the ionic initiator to the hydrogel monomer or the prepolymer thereof is preferably in a range of about 0.5 to about 15 weight percent (wt %), and
a ratio of the polysaccharide or the derivatives thereof to the hydrogel monomer or the prepolymer thereof is preferably in a range of about 5 to about 60 wt %.

2. The composition of claim 1, further comprising a crosslinking agent.

3. The composition of claim 2, wherein a ratio of the crosslinking agent to the hydrogel monomer or the prepolymer thereof is preferably less than about 30 wt %.

4. The composition of claim 2, wherein the crosslinking agent is selected from a group consisting of a divinyl monomer, a prepolymer thereof and bisacrylamide.

5. The composition of claim 1, wherein the ionic initiator is selected from a group consisting of ammonium persulfate, potassium persulfate, 2,2′-azobis(2-methylprionamidine) dihydrochloride and the mixture thereof.

6. The composition of claim 1, wherein the hydrogel monomer or the prepolymer thereof is selected from a group consisting of polymethacrylic acid and ester derivatives thereof containing hydroxyl group, polyacrylamide, polymethacrylamide and derivatives thereof, poly-N-vinyl-2-pyrrolidone, polyvinyl alcohol and the mixture thereof.

7. The composition of claim 1, wherein the polysaccharide is selected from a group consisting of alginic acid, chitin, chitosan, collagen, and hyaluronic acid and the mixture thereof.

8. The composition of claim 1, further comprising an acidic or alkaline substance, wherein the acidic or alkaline substance provides the polysaccharide or the derivatives thereof, lacking charges, with charges.

9. The composition of claim 8, wherein the acidic substance is an organic or inorganic acid.

10. The composition of claim 8, wherein the alkaline substance is an organic or inorganic alkali.

11. A method for preparing composite hydrogel microparticles containing polysaccharide or derivatives thereof, comprising:
performing a dispersion polymerization by mixing an ionic initiator, a hydrogel monomer or a prepolymer thereof, and a polysaccharide or derivatives thereof with water; and
reacting at a temperature of about 10° C. to 100° C. for about 1 to 5 hours;
wherein the ionic initiator provides the hydrogel monomer or the prepolymer thereof with a first charge and the polysaccharide or the derivatives thereof with a second charge, the first charge is opposite the second charge, a ratio of the ionic initiator to the hydrogel monomer or the prepolymer thereof is preferably in a range of about 0.5 to about 15 weight percent (wt %), and a ratio of the polysaccharide or the derivatives thereof to the hydrogel monomer or the prepolymer thereof is preferably in a range of about 5 to about 60 wt %.

12. The method of claim 11, further comprising a crosslinking agent.

13. The method of claim 12, wherein a ratio of the crosslinking agent to the hydrogel monomer or the prepolymer thereof is preferably less than about 30 wt %.

14. The method of claim 12, wherein the crosslinking agent is selected from a group consisting of a divinyl monomer, a prepolymer thereof and bisacrylamide.

15. The method of claim 11, wherein the ionic initiator is selected from a group consisting of ammonium persulfate, potassium persulfate, 2,2′-azobis(2-methylprionamidine) dihydrochloride and the mixture thereof.

16. The method of claim 11, wherein the hydrogel monomer or the prepolymer thereof is selected from a group consisting of polymethacrylic acid and ester derivatives thereof containing hydroxyl group, polyacrylamide, polymethacrylamide and derivatives thereof, poly-N-vinyl-2-pyrrolidone, polyvinyl alcohol and the mixture thereof.

17. The method of claim 11, wherein the polysaccharide is selected from a group consisting of alginic acid, chitin, chitosan, collagen, and hyaluronic acid and the mixture thereof.

18. The method of claim 11, wherein the composite hydrogel microparticles containing polysaccharide or the derivatives thereof is in form of a liquid, latex, gel, and after drying, a powder or film.

19. The method of claim 11, wherein the composite hydrogel microparticles containing the polysaccharide or the derivatives thereof is applied in wound dressings, hemostatic materials, and drug controlled release systems.

20. The method of claim 11, further comprising an acidic or alkaline substance, wherein the acidic or alkaline substance provides the polysaccharide or the derivatives thereof, lacking charge, with charge.

21. The method of claim 20, wherein the acidic substance is an organic or inorganic acid.

22. The method of claim 20, wherein the alkaline substance is an organic or inorganic alkali.
